(19) United States
(12) Patent Application Publication
(10) Pub. No.: US 2024/0233892 A1
(43) Pub. Date: Jul. 11, 2024

(54) TECHNIQUES FOR DETECTION, ASSESSMENT, ALERTING, INTERVENTION AND FEEDBACK SYSTEMS FOR BEHAVIORAL AND MENTAL HEALTH PATIENT CRISES

(71) Applicant: Cerebral Inc., Claymont, DE (US)

(72) Inventors: Akshay SWAMINATHAN, Wood Ridge, NJ (US); Ivan LOPEZ-RODRIGUEZ, Valencia, CA (US); Rafael Antonio GARCIAMAR, Tucson, AZ (US); Matthew RUBASHKIN, Oakland, CA (US); Siddheshwar SALVI, New York, NY (US); Thomas MCCLINTOCK, Concord, CA (US); Tyler HEIST, Madison, WI (US)

(21) Appl. No.: 18/407,414

(22) Filed: Jan. 8, 2024

Related U.S. Application Data
(60) Provisional application No. 63/437,927, filed on Jan. 9, 2023.

(51) Int. Cl. G16H 10/60; G06F 40/40; H04L 51/04

(52) U.S. Cl. CPC G16H 10/60; G06F 40/40; H04L 51/04

(57) ABSTRACT

Techniques for crisis message classification and/or training a crisis message classifier are disclosed. In one particular embodiment, the techniques may be realized as a method for crisis message classification comprising the steps of receiving patient data from a patient, classifying the patient data into an output with a crisis message classifier based on a cost ratio, responsive to determining that the output indicates a crisis, sending an alert to a crisis responder through a user interface, and receiving an input from the crisis responder through the user interface.

### CROSS-REFERENCE TO RELATED APPLICATIONS

This patent application claims priority to U.S. Application No. 63/437,927, entitled “Techniques For Detection, Assessment, Alerting, Intervention and Feedback Systems For Behavioral and Mental Health Patient Crises,” filed Jan. 9, 2023, which is hereby incorporated by reference herein in its entirety.

### FIELD OF THE DISCLOSURE

The present disclosure relates generally to text classification and, more particularly, to techniques for classifying messages as indicating a mental health crisis of a patient.

### BACKGROUND OF THE DISCLOSURE

Previous attempts at crisis message classification have taken different approaches, but each has suffered from at least one drawback. For example, in one such attempt, which involved detecting suicide risk using knowledge-aware natural language processing and counseling service data, a knowledge-graph-based model was developed to detect crisis messages. However, the model was limited by being trained on a Cantonese dataset, leveraging a black box neural network model, having a user interface (UI) that is not designed to smoothly integrate into clinical workflows, not including real-time deployment capabilities, and not including real-time feedback.

In another attempt, which involved a just in time crisis response in the form of a suicide alert system for telemedicine psychotherapy settings, a bag-of-words NLP model was developed to detect suicide risk in messages. However, the model suffered from being focused on suicide rather than a broader set of patient crises, used a UI that was not designed to smoothly integrate into clinical workflows, did not include real-time deployment capabilities, and did not include real-time feedback.

In a further attempt, which involved a novel use of natural language processing (NLP) to predict suicidal ideation and psychiatric symptoms in a text-based mental health intervention, an NLP model was built based on unstructured question data to predict suicide risk and psychiatric symptoms. However, the model required that patients respond to a specific question and was therefore not applicable to any chat message, had a UI not designed to smoothly integrate into clinical workflows, did not include real-time deployment capabilities, and did not include real-time feedback.

In an additional attempt, which involved learning from training a machine learning model to detect suicidal risk, a model was built to identify concerning text messages. However, the model had a UI that was not designed to smoothly integrate into clinical workflows, did not include real-time deployment capabilities, did not include real-time feedback, and was focused on suicide rather than a broader set of patient crises.

In a still further attempt, which involved natural language processing and machine learning of electronic health records for prediction of first-time suicide attempts, an NLP model was built to identify suicide attempt risk using retrospective electronic health record (EHR) data. The model leveraged historical EHR data, not real-time message data, was focused on suicide rather than a broader set of patient crises, had a UI that was not designed to smoothly integrate into clinical workflows, did not include real-time deployment capabilities, and did not include real-time feedback.

In view of the foregoing, it may be understood that there are significant problems and shortcomings associated with current crisis message classification technologies. To address these problems and shortcomings, it would be desirable to have a technique to classify crisis messages that uses a diverse and periodically updated dataset, leverages a machine learning model that is not a black box, has a user interface that smoothly integrates into clinical workflows, has real-time deployment capabilities such as working with real-time message data, facilitates real-time feedback from a crisis operator and/or a patient, is applicable to a broad set of patient crises types (i.e., emergencies), and/or does not require that patients respond to a specific question, therefore making the technique applicable to any chat message.

### SUMMARY OF THE DISCLOSURE

Techniques for crisis message classification and/or training a crisis message classifier are disclosed. In one particular embodiment, the techniques may be realized as a method for crisis message classification comprising the steps of receiving patient data from a patient, classifying the patient data into an output with a crisis message classifier based on a cost ratio, responsive to determining that the output indicates a crisis, sending an alert to a crisis responder through a user interface, and receiving an input from the crisis responder through the user interface.

In accordance with other aspects of this particular embodiment, the crisis message classifier includes a trained natural language processing (NLP) model. The method may further comprise re-training the trained NLP model utilizing the input from the crisis responder and the received patient data.

In accordance with additional aspects of this particular embodiment, the crisis message classifier performs a binary classification of the patient data.

In accordance with further aspects of this particular embodiment, the user interface is accessible by the crisis responder through a messaging application.

In accordance with other aspects of this particular embodiment, the patient data is provided by the patient through a chat application.

In accordance with additional aspects of this particular embodiment, the alert includes a graphical element selectable by the crisis responder, and the input includes feedback on the output with the crisis message classifier provided by a crisis operator.

In accordance with further aspects of this particular embodiment, the input includes a message provided by the crisis responder directly to the patient. In this embodiment, the message may be provided to the patient in real-time.

In accordance with other aspects of this particular embodiment, the cost ratio is the ratio of a cost of false positives to a cost of false negatives generated by the crisis message classifier.

In accordance with further aspects of this particular embodiment, the crisis message classifier determining, in real-time, that the output indicates the crisis.

In one particular embodiment, the techniques may be realized as a method for training a crisis message classifier comprising the steps of receiving a plurality of messages, filtering the plurality of messages with a crisis terms filter into a group of messages, labeling the group of messages, and generating a natural language processing (NLP) model included in the crisis message classifier based on a cost ratio.

In accordance with other aspects of this particular embodiment, the method further comprises re-training the NLP model, the re-training including adding the labeled group of messages to a training data set of the NLP model. The re-training the NLP model may include performing cross validation on the training data set. The cross validation may include using term frequency-inverse document frequency (TF-IDF).

In accordance with further aspects of this particular embodiment, the method comprises classifying a new patient message using the crisis message classifier, and sending an alert to a crisis responder through a user interface. The method may further comprise receiving a selection of a graphical element in the user interface by the crisis operator to thereby provide a label for the new patient message.

In accordance with additional aspects of this particular embodiment, the method further comprises re-training the NLP model responsive to the selection.

In accordance with other aspects of this particular embodiment, the method further comprises determining the cost ratio by using a regret-based approach to assess relative costs of false positives and false negatives generated from the crisis message classifier.

In accordance with additional aspects of this particular embodiment, the method further comprises determining the cost ratio as the ratio of a cost of false positives to a cost of false negatives.

In one particular embodiment, the techniques may be realized as an apparatus for crisis message classification comprising at least one processor readable storage medium storing a computer program of instructions configured to be readable by at least one processor for instructing the at least one processor to execute a computer process for performing a method as described above.

In accordance with other aspects of this particular embodiment, the apparatus includes at least one processor readable storage medium storing a computer program of instructions configured to be readable by at least one processor for instructing the at least one processor to execute a computer process for performing a method as described above.

In one particular embodiment, the techniques may be realized as an article of manufacture for crisis message classification, the article of manufacture comprising at least one processor readable storage medium, and instructions stored on the at least one medium, wherein the instructions are configured to be readable from the at least one medium by at least one processor and thereby cause the at least one processor to operate so as to perform a method as described above.

In one particular embodiment, the techniques may be realized as an article of manufacture for training a crisis message classifier, the article of manufacture comprising at least one processor readable storage medium, and instructions stored on the at least one medium, wherein the instructions are configured to be readable from the at least one medium by at least one processor and thereby cause the at least one processor to operate so as to perform a method as described above.

In one particular embodiment, the techniques may be realized as an apparatus for crisis message classification, the apparatus comprising at least one processor configured to execute instructions, and at least one memory, coupled to the at least one processors, configured to provide the at least one processor with the instructions, the at least one processor configured to execute the instructions to perform a method as described above.

In one particular embodiment, the techniques may be realized as an apparatus for training a crisis message classifier, the apparatus comprising at least one processor configured to execute instructions, and at least one memory, coupled to the at least one processors, configured to provide the at least one processor with the instructions, the at least one processor configured to execute the instructions to perform a method as described above.

In one particular embodiment, the techniques may be realized as a system for training a crisis message classifier, the system comprising one or more processors communicatively coupled to a network; wherein the one or more processors are configured to perform a method as described above.

In one particular embodiment, the techniques may be realized as a system for crisis message classification, the system comprising one or more processors communicatively coupled to a network, wherein the one or more processors are configured to perform a method as described above.

In one particular embodiment, the techniques may be realized as at least one processor readable storage medium storing a computer program of instructions configured to be readable by at least one processor for instructing the at least one processor to execute a computer process for performing a method as described above.

The present disclosure will now be described in more detail with reference to particular embodiments thereof as shown in the accompanying drawings. While the present disclosure is described below with reference to particular embodiments, it should be understood that the present disclosure is not limited thereto. Those of ordinary skill in the art having access to the teachings herein will recognize additional implementations, modifications, and embodiments, as well as other fields of use, which are within the scope of the present disclosure as described herein, and with respect to which the present disclosure may be of significant utility.

### BRIEF DESCRIPTION OF THE DRAWINGS

In order to facilitate a fuller understanding of the present disclosure, reference is now made to the accompanying drawings, in which like elements are referenced with like numerals. These drawings should not be construed as limiting the present disclosure, but are intended to be illustrative only.

FIG. 1 shows a functional block diagram of a method for crisis message classification in accordance with an embodiment of the present disclosure.

FIG. 2 shows a functional block diagram of a feedback method in accordance with an embodiment of the present disclosure.

FIG. 3 shows a crisis EMR safety API, machine learning pipeline, and business logic in accordance with an embodiment of the present disclosure.

FIG. 4 shows a diagram for offline training in accordance with an embodiment of the present disclosure.

FIG. 5 shows a diagram of online/active training of a machine learning model in accordance with an embodiment of the present disclosure.

FIG. 6 shows a functional block diagram of a crisis message training and classification method in accordance with an embodiment of the present disclosure.

FIG. 7 shows a functional block diagram of one example of a processing system in accordance with an embodiment of the present disclosure.

FIG. 8 shows a graphical user interface for a crisis response specialists to provide classifier feedback in accordance with an embodiment of the present disclosure.

### DETAILED DESCRIPTION OF EMBODIMENTS

To solve the above-mentioned problems, embodiments of techniques for detection, assessment, alerting, intervention and feedback systems for behavioral and mental health patient crises are described herein. Specifically, embodiments include natural language text processing and model selection for crisis detection and assessment.

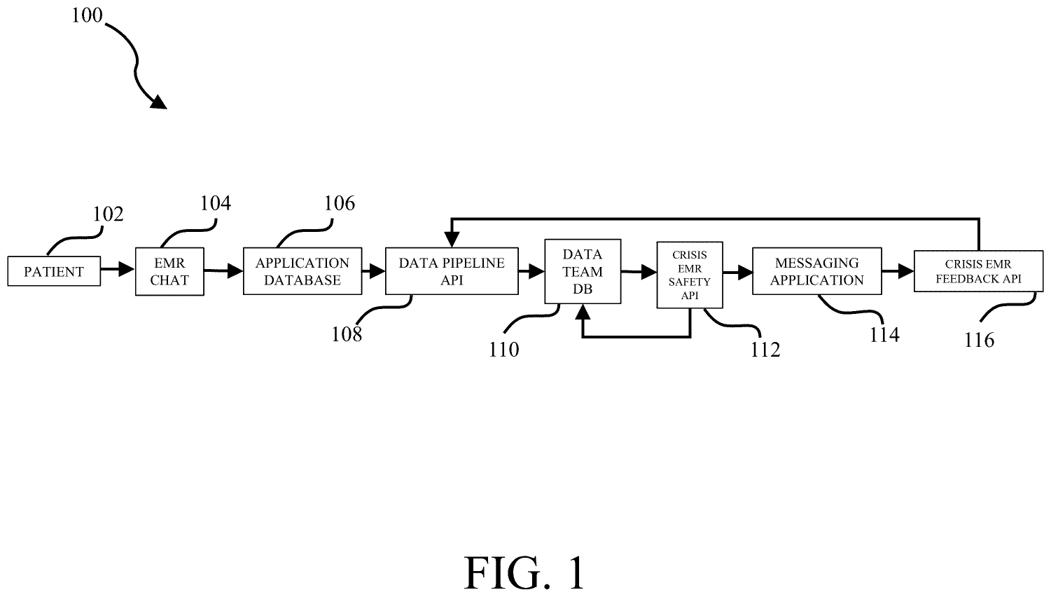

FIG. 1 shows a functional block diagram of a crisis message classification method generally indicated at 100 that illustrates the flow of data through the method 100. The data initially processed by the method 100 may originate from a patient 102 as patient data using an electronic medical record (EMR) chat application 104 (e.g., an application where a patient uses a keyboard or similar data entry device to enter one or more messages in a chat window within a medical provider's website). These messages may then be collected in an application database (DB) 106. Databases described herein, including the application DB 106, may be embodied, in at least one example, as one or more storages or repositories such as a remotely accessible server, a hard drive, and/or a cloud-based repository.

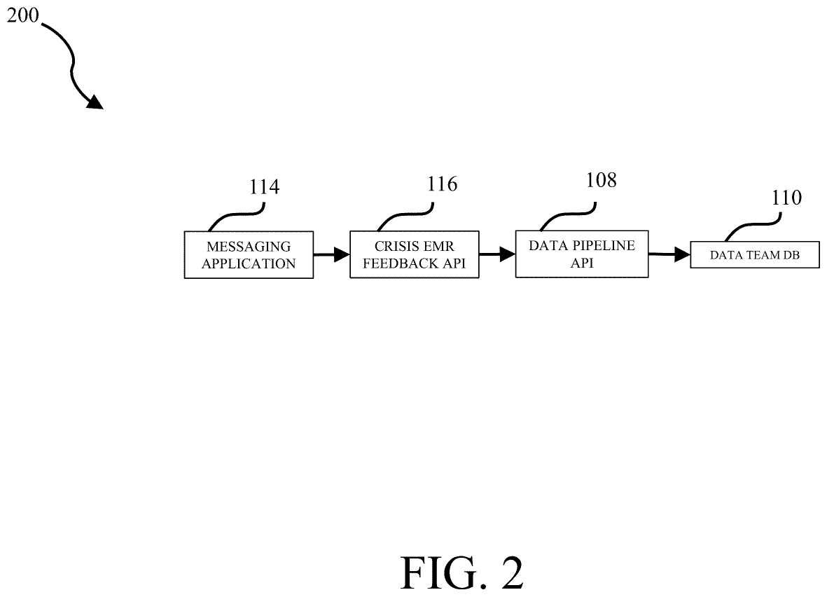

To smoothly connect one programming environment to another, embodiments described herein leverage application programming interfaces (APIs). An API enables a product or a service to communicate with other products and services without having to know exactly how each is implemented, thereby decreasing development time. A data pipeline API 108 (e.g., a REST API) may have access to the application DB 106. The messages may then be pulled by a data team DB 110 through the data pipeline API 108. In one example, the messages may be pulled every 10 minutes. It is understood that this interval may be adjusted to suit a particular need. The crisis EMR safety API 112 may read all these messages and feed them to an NLP model to make predictions. If there are any messages indicating a possible crisis, the crisis EMR safety API 112 may send one or more alerts to a human user interface (e.g., a graphical user interface (GUI)) of a messaging application 114 (e.g., a dedicated space, channel, etc.). The crisis EMR safety API 112 may also communicate with the data team DB 110. The crisis EMR feedback API 116 may communicate with the data pipeline API 108, which is discussed in more detail with respect to FIG. 2.

The messaging application 114, in certain examples, processes data from the data team DB 110 in batches. The crisis EMR feedback API 116, in certain examples, streams data back to the data pipeline 108.

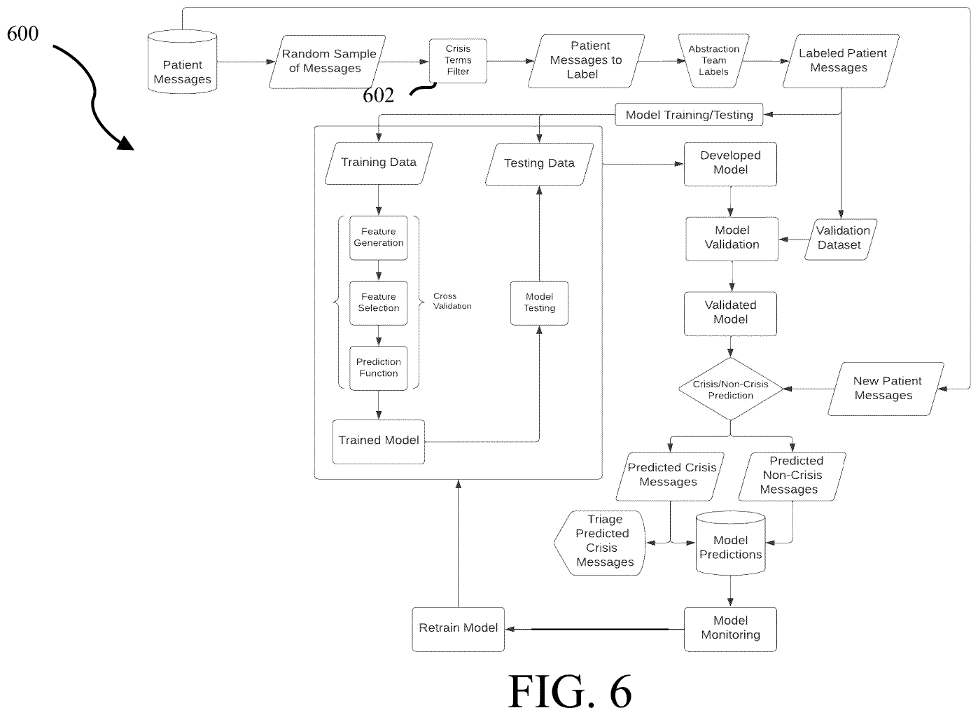

A crisis response team, according to certain examples, may leverage a human user interface in the messaging application 114 to take appropriate action (e.g., initiate crisis protocol) and provide feedback to the crisis EMR feedback API 116. The crisis EMR feedback API 116 may collect all of the data provided by the crisis responders to enable model monitoring and an active learning system (see a more detailed discussion below regarding FIG. 6).

A functional block diagram of a feedback method is generally indicated at 200 in FIG. 2, in which data may flow from crisis responders in the messaging application 114 to a persistence database in the data team DB 110. Crisis responders' interactions with the human user interface may be sent to the crisis EMR feedback API 116 for processing and storage in the persistence database at a predetermined interval via the data pipeline API 108. In one example, the data pipeline API may load the data received from the crisis EMR feedback API 116 into the data team DB 110 once every hour. It is understood that this interval may be adjusted to suit a particular need.

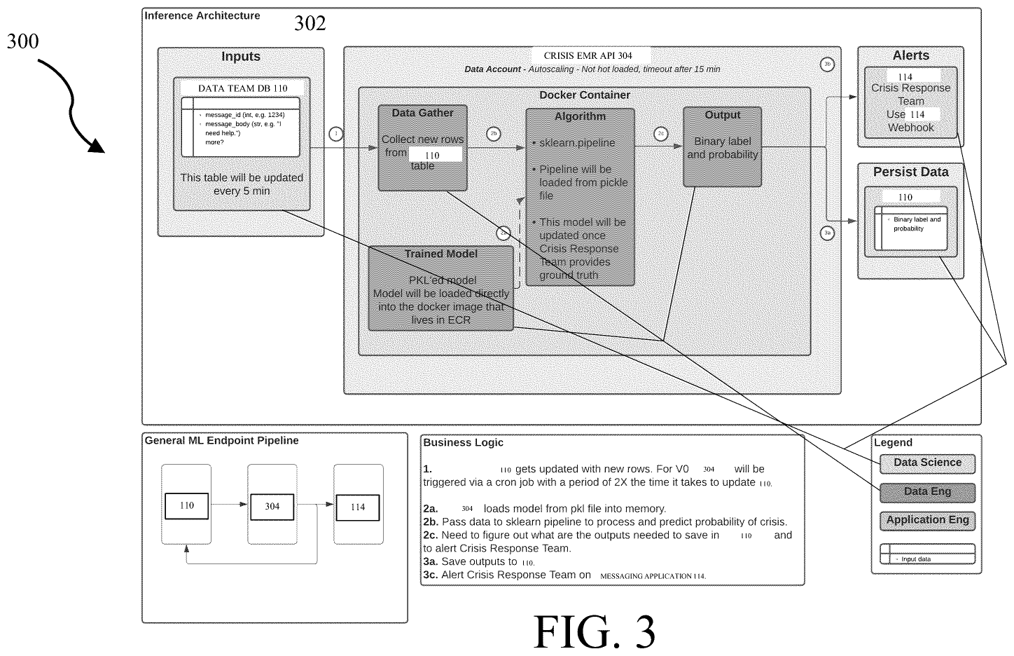

FIG. 3 shows a diagram detailing an inference architecture 302 of the crisis EMR safety API 112. An NLP model and an alerting system may be built into the crisis EMR API 304, which may include the crisis safety API 112 and the crisis EMR feedback API 116. In one example, the crisis EMR API 304 may include the crisis EMR safety API 112 and the crisis EMR feedback API 116. In other examples, the crisis EMR API 304 may include at least one of the crisis EMR safety API 112 and the crisis EMR feedback API 116. The crisis EMR API 304 may run periodically following the business logic provided in FIG. 3. FIG. 3 also shows a general machine learning (ML) pipeline, where the messaging application 114 may receive output from the crisis EMR API 304, which may both receive data from the data team DB 110 and provide the output also back to the data team DB 110.

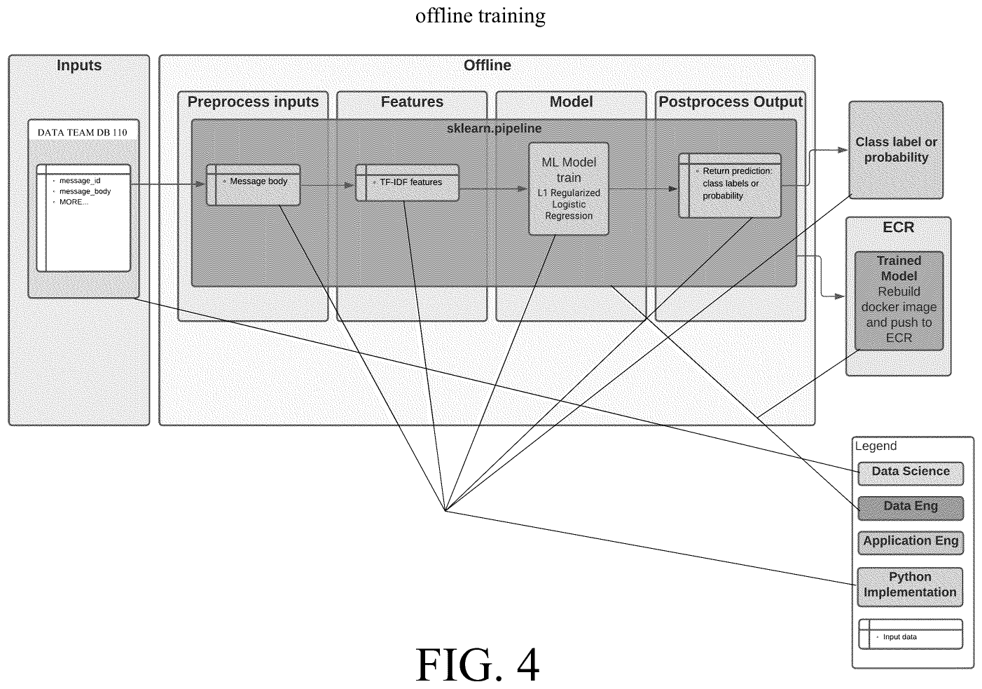

To initially train the NLP model, a method for training a crisis message classifier may be needed. A diagram describing the offline training of the machine learning model is provided in FIG. 4, which shows an example of preprocessing, feature engineering, and a choice of regularization.

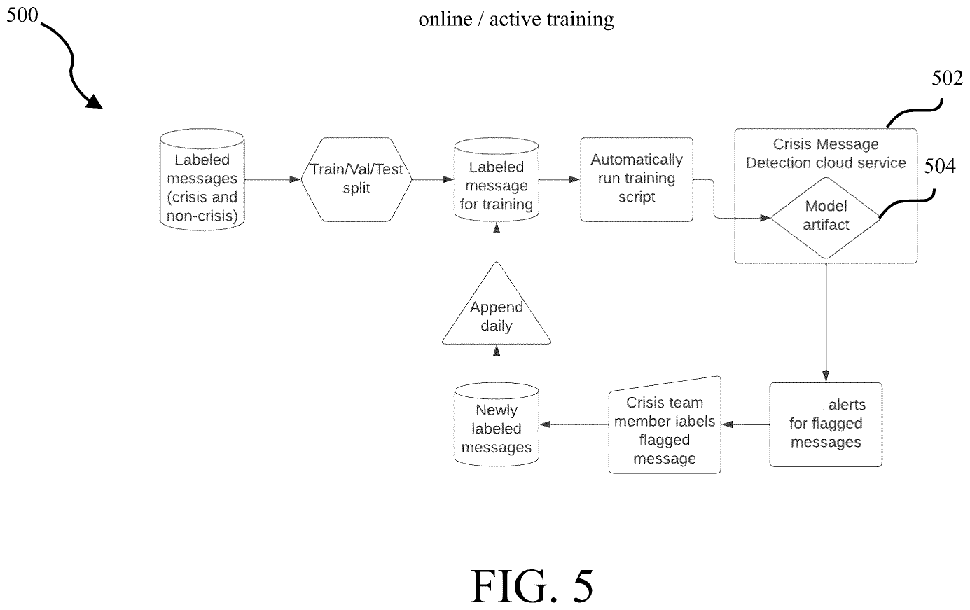

FIG. 5 shows an example of an active learning system generally indicated at 500 for incorporating feedback from crisis team members tasked with responding to flagged messages into the machine learning of the NLP model (after offline training has occurred). A crisis message detection cloud service (CMD) 502 may be included in the active learning system 500. Within the CMD 502, a model artifact 504 may be provided as a piece of software that judges whether individual messages likely reflect a crisis (e.g., using a classifier described above). These messages may be flagged through a messaging application (e.g., the messaging application 114). Crisis response specialists may also monitor these messages and may click a button in a graphical user interface (GUI) to indicate whether the message does or does not indicate a crisis. These responses may be recorded back into a database (e.g., the application database 106) and appended to a database (e.g., the data team DB 110) that may be used for training the model.

The model may be retrained periodically with all labeled data, meaning the training dataset may grow every time it retrains, yielding a slightly better (i.e., more accurate) model each time. In this way, the model may actively incorporate feedback and learn from data labeled by the crisis response specialists.

Putting the offline and online training methods together as an illustrative example, a method for crisis message classification 600 including a method for crisis message classification, as described above, and a method for training a crisis message classifier, as described above, is provided in FIG. 6.

The source of the patient data for the offline training of the system that embodies, performs, executes, or otherwise implements the method for crisis message classification 600 may be a collection of patient messages (e.g., from a patient 102 among other patients). The collection may include, as an example, 5,000-10,000 patient messages; however it is to be understood that other ranges of patient messages may be suitable as well in certain examples, including variation in individual message file size. The patient messages may be received via a chat system (e.g., the EMR chat 104) available on a website or a mobile application of a medical provider. This allows patients to connect with their care team on a variety of topics such as appointment rescheduling, medications, and more. It also serves as a form of contact for patients who are experiencing a crisis.

To develop the NLP model used in the system 600, messages in a database (e.g., the application database 106) that were sent by patients via the chat system within a particular date range may be used. The messages may be received, for example, over the range/course of one year. Messages sent by patients over the subsequent year may be used for model validation. Patient messages selected for model development and validation may then be run through a crisis terms filter 602, and the messages that pass the filter may be considered.

Low event rates may be considered—for example, crisis messages in the database may represent approximately 2% of all patient messages in the database for the first year described above. With a low event rate, one may need to manually label an extremely large and impractical amount of patient messages to create a training dataset that contains a representative sample of all crisis messages. This may be a costly and time consuming task. In order to reduce the load of manual labeling, the crisis terms filter 602 may contain 275 common words and phrases found in crisis text messages. The crisis terms filter 602 may contain words and phrases such as “feel terrible,” “hopelessness,” and “negative thoughts.” Additionally, the crisis terms filter 602 and patient messages may be lemmatized. As a result, words like “stab,” “stabbed,” and “stabbing” may all turn into “stab.”

The crisis terms filter 602, in at least one example, may be constructed by combining one or more different sources of data. In an example, three sources of data may be used. The first source may be created via a literature review of articles attempting to identify words and phrases that signal suicide risk. Terms found during the literature review may be added to the terms list. The second data source may come from a dataset of existing words and phrases that has been validated in similar studies on imminent harm classifiers and social media applications. The third source may involve feeding randomly selected messages from the database through the growing terms filter and finding crisis messages that were filtered out. These crisis messages may then be reviewed and words and phrases may be added to the crisis terms list to prevent similar crisis messages from being excluded in the future. In at least one example, the structure for implementing the algorithm and/or function of the crisis terms filter 602 may be a computer processor. It is understood that methods and systems and other embodiments described herein may include crisis terms filters that are constructed using different, but suitable other sources than those described above.

Keeping with the examples above, feature generation may be achieved via TF-IDF (term frequency-inverse document frequency). TF-IDF is typically a numerical statistic that assigns scores to words or phrases. Each score may attempt to represent the importance of a word or phrase to a document in a corpus. For the NLP model in the system implementing the method for crisis message classification 600, TF-IDF scores may be calculated for single words and phrases of up to 5 words. Using this technique to generate features may result in a feature matrix with very high dimensionality. This is a common problem in natural language processing because many of these features are sparse. A feature is considered sparse when values are missing High amounts of sparse features can impact the performance of machine learning models. To remove sparse features, a sparsity value may be selected that brings the total misclassification cost function to a minimum during model training.

In some embodiments, other algorithms are used to create features to be used in the model. In one example, the word vector representation algorithm word2vec is used. Using a different feature generation algorithm may require the model to be retrained on the vector representations of the words. Still further, in other embodiments, advanced deep learning models including but not limited to BERT or GPT are leveraged to create these feature representations of words.

The NLP model in an example may be trained on 60% of the labeled data (i.e., 721 messages) and tested on the remaining 40% (i.e., 481 messages). Feature selection may be achieved through L1-regularized logistic regression (i.e., LASSO Regression) with the regularization parameter tuned through 10-fold cross-validation to minimize total misclassification of the cost function: Total cost=[(cost of FP)*(# of FP)+(cost of FN)*(# of FN)/(Total N observations), where “FP” indicates false positive and “FN” indicates false negative. Here, “cost” refers not to monetary cost, but rather the real-world utilities corresponding to FPs and FNs. In this case, the cost of an FP may be the cost of incorrectly flagging a non-crisis message (e.g., increased review burden on crisis specialists) and the cost of an FN may be the cost of incorrectly missing a true crisis message (e.g., delayed intervention for a patient in distress).

It is important that the probability threshold for these classification models be selected to reflect the end user's relative tolerance for FPs vs. FNs. To this end, relevant stakeholders may be surveyed (e.g., clinicians, operations leaders, data scientists) using a regret-based approach to assess the relative costs of false positives and false negatives. Such a survey presents a scenario with, for example, 1000 patient messages to be classified by an imperfect prediction model and asking a user to quantify how many patient messages they would be willing to review manually to avoid a false positive and false negative. The responses can be used to determine the relative undesirability of false positives and false negatives.

In another example, six user and developer responses may be collected. All six respondents may be aligned, categorized, or otherwise sorted according to their preference that missing a true crisis message (i.e., false negative) is worse than falsely flagging a non-crisis message (i.e., false positive). The results of the cost ratio survey may suggest using a false positives to false negatives cost ratio of 20:1. This means that the model returning a false negative is 20 times more costly than returning a false positive.

The probability threshold of the CMD 502 may be selected by finding the threshold that produces the minimum misclassification cost. Parameters of the machine learning, sparsity, and the probability threshold that bring the total misclassification cost function to a minimum may be used for model testing and deployment.

The CMD 502 may be a crisis message classifier. For each unit of observation, defined in this example as a message, CMD 502 may output a probability between 0 and 1 indicating the probability that the given message is a crisis message. The probability threshold may be used to label whether a message is a crisis or non-crisis (i.e., a binary classification). Messages with predicted probabilities greater than the probability threshold may be labeled as crisis messages, and messages with predicted probabilities below the probability threshold may be labeled as non-crisis. Applying a probability threshold to the CMD 502 may convert the model from a probability prediction model into a binary classifier.

To evaluate and validate performance of the classifier in at least one example, sensitivity, specificity, positive predictive value (PPV), negative predictive value (NPV), and total misclassification cost may be used to evaluate the performance of the CMD 502 in training and testing phases.

Throughout the embodiments described herein, the classifier (e.g., the CMD 502) may generate its output(s) or other decisions in real-time or near real-time. Processing may be completed in real-time when, in at least one example, the processing occurs so as to not otherwise require a user to wait for the process (e.g., classification with the CMD 502) to finish before moving on to another task. In certain examples, processing being completed in near real-time may correspond to the processing being performed instantaneously, if not for the laws of physics, physical limitations of computer processors, available bandwidth, and so forth.

Furthermore, after training, the CMD 502 may be used to generate predictions on a randomly selected sample of, for example, 200 unlabeled patient messages sent through the second subsequent year described above as a validation dataset. These 200 messages may not be included in the training or testing datasets for the CMD 502. The CMD 502 may make two types of predictions: “crisis” and “non-crisis.” These 200 messages may then be manually labeled as either “crisis” or “non-crisis,” and the performance of the CMD 502 may be measured to validate the model. In at least one example, the 200 messages may be automatically labeled. In yet another example, the 200 messages may be labeled using a combination of manual and automated processes.

Messages that are tagged/flagged as crisis messages may be surfaced to the crisis response team via a messaging application (e.g., the messaging application 114). Crisis responders may get notified so they can determine if the flagged messages indicate true crises or not and initiate the appropriate crisis response protocol when required.

The NLP model, in certain examples, may be hosted as a Function as a Service (FaaS) using a Python runtime. This function may run on a schedule every 10 minutes and perform the following actions: first, read new messages received from patients using the Snowflake Connector for Python; then run messages through the NLP model to make predictions, where the NLP model in this example may be a scikit-learn pipeline that preprocesses the data and makes predictions; and then positive predictions (i.e., alerts) may be surfaced to a channel of the messaging application (e.g., a Slack channel using the Slack Web API built into the Python Slack SDK).

The alerts may be posted in the channel using a bot (e.g., a Slack Bot). Each alert may appear as new message containing, for example: message_body, message_ID, received timestamp, patient ID, and link to patient chart. Such alerts may, in at least some embodiments, be stored in a table within the data team DB 110, as shown in the inference architecture 302 in FIG. 3.

Crisis responders may react to these alerts in a user interface using emojis, for example, and reply to threads therein to indicate that they are taking care of an alert. This prevents multiple people from working on the same alert. Responders may use the content of the message, previous messages and/or the patient's chart to determine if a true crisis is occurring. Responders may directly chat with the patient in real-time to gather more information. Once the responders have determined if the alert is a true crisis or not, they may reply to the alert in a thread indicating whether a crisis did or did not occur, the reason for the decision, and actions taken.

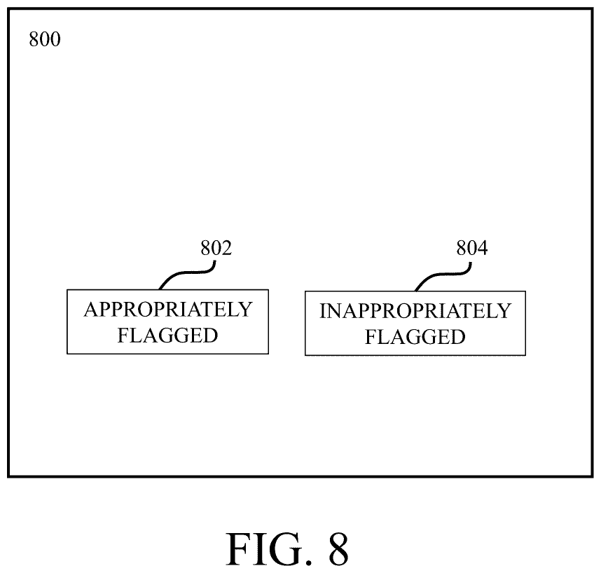

Additionally, the alert, in an example, may contain two GUI buttons labeled “Appropriately flagged” and “Inappropriately flagged,” as shown in FIG. 8. By pushing one of these buttons, the crisis responder may indicate that “the NLP model (in)appropriately flagged this messages as a crisis.” This button press may be an alert response recorded in the application database 106 and/or other databases. By associating the alert response with the original message, production data including additional labeled data may be produced.

The production data may have the same format as the original data used to train the NLP model. Thus, one may automate a system to append the production data to the original training data and train a new model. This new model may perform better (up to the noise limit) on the validation dataset (i.e., the held-out set), hence automatically updating the crisis detection system with this new model. In this way, the system may actively learn from messages labeled in near real time, and improve over time as new crisis are found.

At this point it should be noted that crises message processing in accordance with the present disclosure as described above may involve the processing of input data and the generation of output data to some extent. This input data processing and output data generation may be implemented in hardware or software. For example, specific electronic components may be employed in a cloud-based server or similar or related circuitry for implementing the functions associated with crises message classifiers, programs, interfaces, systems, and so forth in accordance with the present disclosure as described above. Alternatively, one or more processors operating in accordance with instructions may implement the functions associated with implementing and/or training a crises message classifier (e.g., the CMD 502) in accordance with the present disclosure as described above. If such is the case, it is within the scope of the present disclosure that such instructions may be stored on one or more non-transitory processor readable storage media (e.g., a magnetic disk or other storage medium), or transmitted to one or more processors via one or more signals embodied in one or more carrier waves.

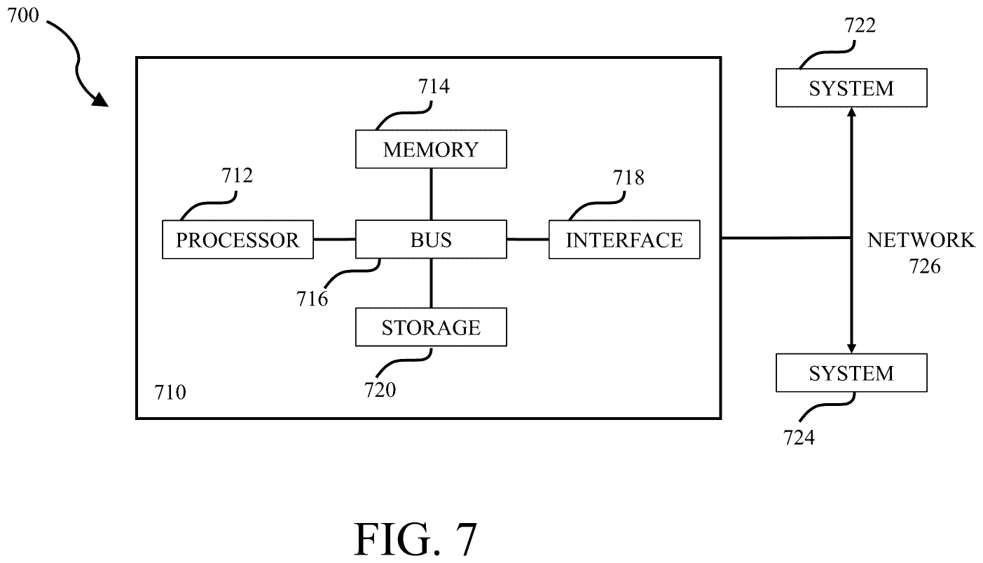

FIG. 7 is a block diagram of a distributed computer system 700, in which various aspects and functions discussed above may be practiced. The distributed computer system 700 may include one or more computer systems. For example, as illustrated, the distributed computer system 700 may include three computer systems 710, 722, and 724. As shown, the computer systems 710, 722, and 724 may be interconnected by, and may exchange data through, a communication network 726. The network 726 may include any communication network through which computer systems may exchange data. To exchange data via the network 726, the computer systems 710, 722, and 724 and the network 726 may use various methods, protocols and standards including, among others, token ring, Ethernet, Wireless Ethernet, Bluetooth, radio signaling, infra-red signaling, TCP/IP, UDP, HTTP, FTP, SNMP, SMS, MMS, SS7, JSON, XML, REST, SOAP, CORBA HOP, RMI, DCOM and Web Services.

According to some embodiments, the functions and operations discussed for controlling one or more crises message classifiers (e.g., similar to the CMD 502) with a logic process (e.g., similar to the method 100 in FIG. 1) can be executed on computer systems 710, 722, and 724 individually and/or in combination. For example, the computer systems 710, 722, and 724 may support, for example, participation in a collaborative network. In one alternative, a single computer system (e.g., 710) can perform a graceful degradation logic process. The computer systems 710, 722, and 724 may include personal computing devices such as cellular telephones, smart phones, tablets, “fablets,” etc., and may also include desktop computers, laptop computers, etc.

Various aspects and functions in accordance with embodiments discussed herein may be implemented as specialized hardware or software executing in one or more computer systems including the computer system 710 shown in FIG. 7. In one embodiment, computer system 710 may be a personal computing device specially configured to execute the processes and/or operations discussed above. As depicted, the computer system 710 may include at least one processor 712 (e.g., a single core or a multi-core processor), a memory 714, a bus 716, input/output interfaces (e.g., 718) and storage 720. The processor 712, which may include one or more microprocessors or other types of controllers, can perform a series of instructions that manipulate data. As shown, the processor 712 may be connected to other system components, including a memory 714, by an interconnection element (e.g., the bus 716).

The memory 714 and/or storage 720 may be used for storing programs and data during operation of the computer system 710. For example, the memory 714 may be a relatively high performance, volatile, random access memory such as a dynamic random access memory (DRAM) or static memory (SRAM). In addition, the memory 714 may include any device for storing data, such as a disk drive or other non-volatile storage device, such as flash memory, solid state, or phase-change memory (PCM). In further embodiments, the functions and operations discussed with respect to controlling one or more crises message classifiers, human interfaces, cloud services, and so forth, can be embodied in an application that is executed on the computer system 710 from the memory 714 and/or the storage 720. For example, the application can be made available through an “app store” for download and/or purchase. Once installed or made available for execution, computer system 710 can be specially configured to execute the methods described herein.

The computer system 710 may also include one or more interfaces 718 such as input devices, output devices and combination input/output devices. The interfaces 718 may receive input (e.g., the EMR chat 104 receiving messages from the patient 102), provide output, or both. The storage 720 may include a computer-readable and computer-writeable nonvolatile storage medium in which instructions are stored that define a program to be executed by the processor. The storage system 720 also may include information that is recorded, on or in, the medium, and this information may be processed by the application. A medium that can be used with various embodiments may include, for example, optical disk, magnetic disk or flash memory, SSD, NVMe, among others. Further, aspects and embodiments are not to a particular memory system or storage system.

In some embodiments, the computer system 710 may include an operating system that manages at least a portion of the hardware components (e.g., input/output devices, touch screens, cameras, etc.) included in computer system 710. One or more processors or controllers, such as processor 712, may execute an operating system which may be, among others, a Windows-based operating system (e.g., Windows NT, ME, XP, Vista, 7, 8, 10, 11, or RT) available from the Microsoft Corporation, an operating system available from Apple Computer (e.g., MAC OS, including System X), one of many Linux-based operating system distributions (for example, the Enterprise Linux operating system available from Red Hat Inc.), a Solaris operating system available from Sun Microsystems, or a UNIX operating systems available from various sources. Many other operating systems may be used, including operating systems designed for personal computing devices (e.g., iOS, Android, etc.) and embodiments are not limited to any particular operating system.

The processor and operating system together may define a computing platform on which applications (e.g., “apps” available from an “app store”) may be executed. Additionally, various functions for producing and processing optical signals may be implemented in a non-programmed environment (for example, documents created in HTML, XML or other format that, when viewed in a window of a browser program, render aspects of a GUI or perform other functions). Such a GUI is shown in FIG. 8.

When a crisis message classifier (e.g., the CMD 502) provides an output (e.g., a binary crisis classification), an alert may be sent to a crisis response specialist as described above for classifier feedback. FIG. 8 illustrates an exemplary graphical user interface 800 including a first button 802 and a second button 804. The buttons 802, 804 may be user-selectable graphic elements. By clicking/touching/selecting the first button 802, the crisis response specialist may assess and confirm the classifier output as accurate (e.g., flagged appropriately). By clicking the second button 804, the specialist or crisis operator may indicate the opposite, that the classifier has not properly classified the new patient message prompting the alert. Clicking either button 802, 804 may provide feedback that can be used to further train and improve the crisis message classifier. In other words, clicking the first button 802 or the second button 804 may provide a label as a confirmation for the prediction of the new patient message prompting the alert.

It is understood that the GUI 800 is merely exemplary and other graphical user interfaces may be utilized by, for example, including additional buttons for other types of feedback, larger or smaller graphic elements, and so forth. In another example, two GUI buttons that each trigger a separate program function can be combined into a GUI knob, slider, or similar element that can select between more than one program function.

Further, various embodiments may be implemented as programmed or non-programmed components, or any combination thereof. Various embodiments may be implemented in part as MATLAB functions, scripts, and/or batch jobs. Thus, the described and enabled embodiments are not limited to a specific programming language and any suitable programming language could also be used.

Although the computer system 710 is shown by way of example as one type of computer system upon which various functions may be practiced, aspects and embodiments are not limited to being implemented on the computer system, shown in FIG. 7. Various aspects and functions may be practiced on one or more computers or similar devices having different architectures or components than that shown in FIG. 7.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Further, although the present disclosure has been described herein in the context of at least one particular implementation in at least one particular environment for at least one particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

1. A method for crisis message classification comprising the steps of:
receiving patient data from a patient;
classifying the patient data into an output with a crisis message classifier based on a cost ratio;
responsive to determining that the output indicates a crisis, sending an alert to a crisis responder through a user interface; and
receiving an input from the crisis responder through the user interface.

2. The method of claim 1, wherein the crisis message classifier includes a trained natural language processing (NLP) model.

3. The method of claim 2, further comprising re-training the trained NLP model utilizing the input from the crisis responder and the received patient data.

4. The method of claim 1, wherein the crisis message classifier performs a binary classification of the patient data.

5. The method of claim 1, wherein the user interface is accessible by the crisis responder through a messaging application.

6. The method of claim 1, wherein the patient data is provided by the patient through a chat application.

7. The method of claim 1, wherein the alert includes a graphical element selectable by the crisis responder, and the input includes feedback on the output with the crisis message classifier provided by a crisis operator.

8. The method of claim 1, wherein the input includes a message provided by the crisis responder directly to the patient.

9. The method of claim 1, wherein the cost ratio is the ratio of a cost of false positives to a cost of false negatives generated by the crisis message classifier.

10. The method of claim 1, further comprising:
the crisis message classifier determining, in real-time, that the output indicates the crisis.

11. An apparatus for crisis message classification comprising:
at least one processor readable storage medium storing a computer program of instructions configured to be readable by at least one processor for instructing the at least one processor to execute a computer process for performing a method, the method comprising:
receiving patient data from a patient;
classifying the patient data into an output with a crisis message classifier based on a cost ratio;
responsive to determining that the output indicates a crisis, sending an alert to a crisis responder through a user interface; and
receiving an input from the crisis responder through the user interface.

12. The apparatus of claim 11, wherein the crisis message classifier includes a trained natural language processing (NLP) model.

13. The apparatus of claim 12, wherein the method further comprises re-training the trained NLP model utilizing the input from the crisis responder and the received patient data.

14. The apparatus of claim 11, wherein the crisis message classifier performs a binary classification of the patient data.

15. The apparatus of claim 11, wherein the user interface is accessible by the crisis responder through a messaging application.

16. The apparatus of claim 11, wherein the patient data is provided by the patient through a chat application.

17. The apparatus of claim 11, wherein the alert includes a graphical element selectable by the crisis responder, and the input includes feedback on the output with the crisis message classifier provided by a crisis operator.

18. The apparatus of claim 11, wherein the input includes a message provided by the crisis responder directly to the patient.

19. The apparatus of claim 11, wherein the cost ratio is the ratio of a cost of false positives to a cost of false negatives generated by the crisis message classifier.

20. At least one processor readable storage medium storing a computer program of instructions configured to be readable by at least one processor for instructing the at least one processor to execute a computer process for performing a method, the method comprising:
receiving patient data from a patient;
classifying the patient data into an output with a crisis message classifier based on a cost ratio;
responsive to determining that the output indicates a crisis, sending an alert to a crisis responder through a user interface; and
receiving an input from the crisis responder through the user interface.
